(19) United States
(12) Patent Application Publication
(10) Pub. No.: US 2020/0268250 A1
(43) Pub. Date: Aug. 27, 2020

(54) DEVICE AND SYSTEM FOR PROVIDING PHYSIOLOGICAL DATA MONITORING OF PATIENTS

(71) Applicant: Drägerwerk AG & Co. KGaA, Lübeck (DE)

(72) Inventor: Barry M. YOMTOV, Marblehead, MA (US)

(21) Appl. No.: 16/649,880

(22) PCT Filed: Sep. 26, 2018
(86) PCT No.: PCT/US18/52803

§ 371 Date: Mar. 23, 2020

Related U.S. Application Data
(60) Provisional application No. 62/564,104, filed on Sep. 27, 2017.

(51) Int. Cl. A61B 5/00; A61B 5/0408

(52) U.S. Cl. CPC A61B 5/0006; A61B 5/04085; A61B 2560/0443; A61B 5/0024; A61B 5/04087

(57) ABSTRACT

A physiological monitoring system includes an electrocardiogram (ECG) module and a main module connected by a first communication connection that is wireless. The main module receives the first physiological data from the ECG module using the first communication connection. The main module stores the physiological data and transmits alerts using both wireless or wired communication connections. The ECG module can identify one or more abnormal conditions of the patient, and transmit in real-time results of the analysis to the main module using the first communication connection and/or transmit alerts or alarms directly to the patient's skin. Additionally, a detachable pre-cordial electrode array can be added that includes a second plurality of electrodes worn in a precordial location of the patient to gather second physiological data and transmits the second physiological data to the ECG module or the main module.

### TECHNICAL FIELD

The subject matter of the present disclosure relates generally to patient monitoring devices used for collecting physiological data.

### BACKGROUND

Patient monitoring devices are essential medical devices that provide vital physiological data to clinicians and caregivers for the care of patients. However, patient monitoring can present challenges both inside and outside hospital environments. For example, patients that are admitted to a healthcare facility may require continuous physiological monitoring, and this continual physiological monitoring can be a data intensive task. These challenges can be accentuated when the patients being monitored are ambulatory (i.e., moveable) because the devices used for monitoring patient parameters are also required to be ambulatory (i.e., moveable) so that patients are not confined to a particular bed or to a particular care unit.

Healthcare facilities have been outfitted with a network of wireless access points that enable wireless communication between a central monitoring station and patient monitoring devices. With the implementation of wireless data communications, the ability to remotely monitor patients has expanded the use of ambulatory patient monitoring devices used for monitoring physiological data. However, even with ambulatory patient monitoring devices, there are still challenges regarding device performance, patient compliance, and quality of life for patients when these devices are used over long periods of time.

Additionally, with hospital enterprises now expanding to alternate lower acuity care settings such as rehabilitation centers, the requirements for continuous physiological data monitoring has become more focused on quality of life when considering patients are more mobile (ambulatory) and the physiological monitors are operating 24 hours a day.

Thus, it would be advantageous and an improvement over conventional patient monitoring systems to provide a patient worn physiological monitoring device or system for ambulatory patients that improve device performance, patient compliance, quality of life for patients, and overall patient outcomes during ambulatory activity associated with recuperation and rehabilitation.

### SUMMARY

In an embodiment described in the present disclosure, a physiological monitoring system for providing monitoring of a patient includes an electrocardiogram (ECG) module having an ECG microcontroller and a first plurality of electrodes worn on the patient; and a main module detachably worn by the patient and connected to the ECG module by a first communication connection. The ECG microcontroller is coupled to the first plurality of electrodes for receiving first physiological data gathered by the first plurality of electrodes, and the main module is configured to receive the first physiological data from the ECG module using the first communication connection.

The ECG microcontroller is further configured to analyze the first physiological data gathered by the first plurality of electrodes, identify one or more abnormal conditions of the patient, and transmit in real-time results of the analysis of the first physiological data and the identified abnormal conditions to the main module using the first communication connection. The first communication connection is a wireless communication connection.

In an embodiment described in the present disclosure, the physiological monitoring system further comprising a detachable precordial electrode array including a second plurality of electrodes worn in a precordial location of the patient proximate to the ECG module. The second plurality of electrodes are configured to gather second physiological data, and the ECG module is connected to the detachable precordial electrode array by a second communication connection.

The ECG microcontroller is further configured to analyze the second physiological data gathered by the second plurality of electrodes, identify one or more abnormal conditions of the patient, and transmit in real-time results of the analysis of the second physiological data and the identified abnormal conditions to the main module using the first communication connection. The first communication connection is a wireless communication connection, and the second communication is a wired communication connection.

In an embodiment described in the present disclosure, the main controller is further configured to transmit in real-time results of the analysis of the first physiological data or the second physiological data and the identified abnormal conditions using a first wireless protocol of the communication interface, or store the first physiological data or the second physiological data and the identified abnormal conditions in the on-board memory when the main module is unable to transmit the in real-time using the first wireless protocol of the communication interface.

The main controller is further configured transmit in real-time results of the analysis of the first physiological data or the second physiological data and the identified abnormal conditions using a second wireless protocol of the communication interface if a significant physiological event is identified from the analysis of the first physiological data or the second physiological data. The first wireless protocol is in accordance with WIFI or Bluetooth, whereas the second wireless protocol is in accordance with a cellular network.

In an embodiment described in the present disclosure, the detachable precordial electrode array is connected to the main module by a third communication connection for transmitting the second physiological data gathered by the second plurality of electrodes to the main module. The main controller is configured to receive the second physiological data from the ECG module using the third communication connection, wherein the third connection is a wired connection.

In an embodiment described in the present disclosure, the detachable precordial electrode array includes a plurality of electrodes worn in a precordial location of the patient, and a communication connection for transmitting the physiological data gathered by the plurality of electrodes. The plurality of electrodes are formed in a flexible material integrated as a patch and having a bottom surface that is attachable to the patient for gathering the physiological data.

In an embodiment described in the present disclosure, a patch with the first plurality of electrodes is detachable from the data acquisition module and disposable, and the data acquisition module is re-useable.

In an embodiment described in the present disclosure, the data acquisition module and the patch with the first plurality of electrodes are integrated, and both the patch and the data acquisition module are disposable.

In an embodiment described in the present disclosure, the ECG module further comprises adjustment slots and a location of each of the first plurality of electrodes is adjustable within the adjustment slots.

In an embodiment described in the present disclosure, the detachable precordial array further comprises adjustment slots and a location of each of the second plurality of electrodes is adjustable within the adjustment slots.

### BRIEF DESCRIPTION OF THE DRAWINGS

FIG. 1 is a block diagram of a main module for physiological monitoring according to an embodiment of the present disclosure;

FIG. 2 is an exemplary algorithm executed by the microcontrollers of the main module according to an embodiment of the present disclosure;

FIG. 3 is a block diagram of a wireless electrocardiogram (ECG) module according to an embodiment of the present disclosure;

FIG. 4 is an exemplary algorithm executed by a microcontroller of the wireless ECG module according to an embodiment of the present disclosure;

FIG. 5 is a diagram of a physiological monitoring system including the wireless ECG module and the main module according to an embodiment of the present disclosure;

FIG. 6 is a diagram of a physiological monitoring system including a precordial electrode array connected to the wireless ECG module according to an embodiment of the present disclosure;

FIG. 7A is a top view of the wireless ECG module according to an embodiment of the present disclosure;

FIG. 7B is side view of the wireless ECG module according to an embodiment of the present disclosure;

FIG. 8A is side view of the connection between the data acquisition module and the electrode patch of the wireless ECG module according to an embodiment of the present disclosure;

FIG. 8B is a bottom view of the electrode connections of the data acquisition module according to an embodiment of the present disclosure;

FIG. 9A is top view of the precordial electrode array with cable and in-line connector according to an embodiment of the present disclosure;

FIG. 9B is a cross-sectional view of the keyed twist lock on the cable of the precordial electrode array according to an embodiment of the present disclosure;

FIG. 10 is a side view of the connection between the data acquisition module and the precordial electrode array according to an embodiment of the present disclosure;

FIGS. 11A and 11B are top views respectively of the adjustable electrode slots on the wireless ECG module and the precordial electrode array;

FIG. 12A is a top view of the wireless ECG module according to an embodiment of the present disclosure;

FIG. 12B is a side view of wireless ECG module showing the embedded circuitry according to an embodiment of the present disclosure;

FIG. 13 is a diagram of a physiological monitoring system including the precordial electrode array connected to the main module according to an embodiment of the present disclosure; and

FIG. 14 is a side view of the connection between the main module and the precordial electrode array according to an embodiment of the present disclosure.

### DETAILED DESCRIPTION

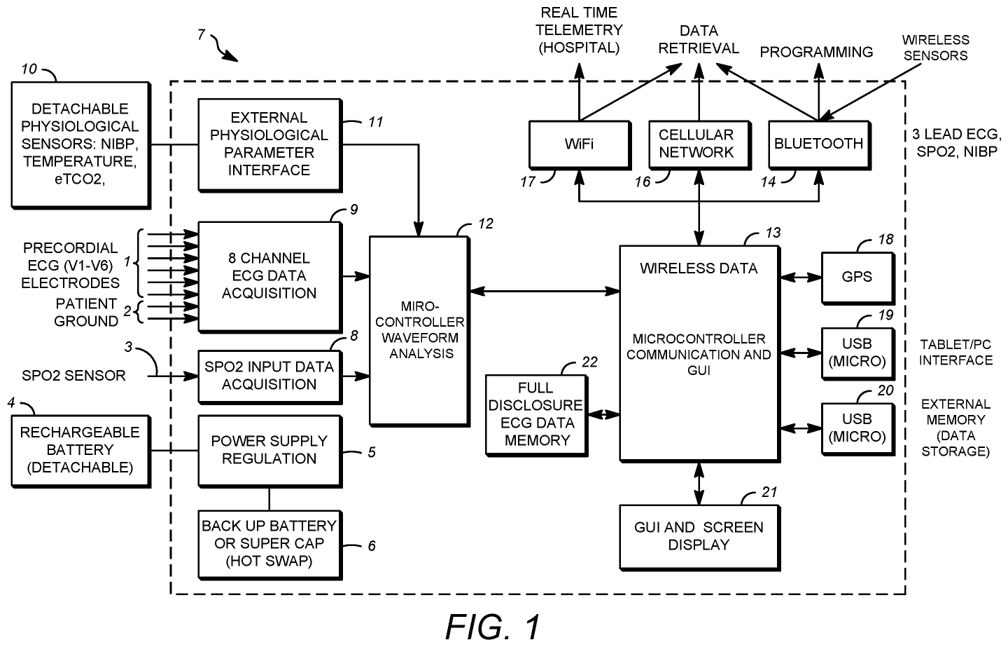

FIG. 1 is a block diagram of a main module for physiological monitoring according to an embodiment of the present disclosure.

As shown in FIG. 1, the main module 7 is attached to several different types of electrodes and sensors known in the art for gathering physiological data related to a patient (e.g., as shown on the left side of FIG. 1). The electrodes and sensors are attached to the main module by, for example, a wired connection. However, the main module 7 can also be connected to wireless sensors using communication interface circuitry for receiving data from and sending data to one or more devices using, for example, a Bluetooth connection 14. The data signals from the electrodes and sensors received by the main module 7 include data related to an electrocardiogram (ECG), non-invasive peripheral oxygen saturation (SpO2), non-invasive blood pressure (NIBP), temperature, and/or tidal carbon dioxide (eTCO2). For example, the data signals related to ECG and SpO2 are received respectively from the precordial ECG electrodes 1 and the SpO2 sensor 3. The data signals received from the precordial ECG electrodes 1 and the SpO2 sensor 3 are, for example, analog signals. The data signals from the precordial ECG electrodes 1 are input to the ECG data acquisition circuit 9 and the SpO2 data signal from the SpO2 sensor 3 is input to the SpO2 data acquisition circuit 8. Both the ECG data acquisition circuit 9 and the SpO2 data acquisition circuit 8 include amplifying and filtering circuitry as well as analog-to-digital (A/D) circuitry that convert the analog signal to a digital signal using amplification, filtering, and A/D conversion methods known in the art.

The data signals related to NIBP, temperature, and eTCO2 are received from a detachable physiological sensors 10 connected to the main module 7 through an external physiological parameter interface 11. The external physiological parameter interface 11 includes, for example, serial interface circuitry for receiving and processing the data signals related to NIBP, temperature, and eTCO2. The processing performed by the ECG data acquisition circuit 9, the SpO2 data acquisition circuit 8, and external physiological parameter interface 11 produces digital data waveforms, which are passed to a dedicated microcontroller 12 by electrical connection therebetween. The digital data waveforms are analyzed by the microcontroller 12 to identify any abnormal conditions of the patient. The microcontroller 12 analyzes the digital waveforms to identify certain digital waveform characteristics and threshold levels indicative of abnormal conditions of the patient using methods known in the art.

The microcontroller 12 is, for example, a processor, a field programmable gate array (FPGA), an application specific integrated circuit (ASIC), a digital signal processor (DSP), or similar processing device. The microcontroller 12 also includes a memory. The memory is, for example, a random access memory (RAM), a memory buffer, a hard drive, a database, an erasable programmable read only memory (EPROM), an electrically erasable programmable read only memory (EEPROM), a read only memory (ROM), a flash memory, or a hard disk.

The memory stores software or algorithms with executable instructions and the microcontroller 12 can execute a set of instructions of the software or algorithms in association with executing an operation of analyzing the digital data waveforms related to the data signals of the electrodes and sensors 1, 3, and 10 to identify abnormal conditions of the patient.

The results of the analysis by the microcontroller 12 are passed to the microcontroller 13 by an electrical connection between the microcontrollers 12, 13. The microcontroller 13 is, for example, a processor, a field programmable gate array (FPGA), an application specific integrated circuit (ASIC), a digital signal processor (DSP), or similar processing device. The microcontroller 13 also includes a memory. The memory is, for example, a random access memory (RAM), a memory buffer, a hard drive, a database, an erasable programmable read only memory (EPROM), an electrically erasable programmable read only memory (EEPROM), a read only memory (ROM), a flash memory, or a hard disk.

Additionally, the microcontroller 13 includes communication interface circuitry for establishing communication connections with various devices and networks using both wired and wireless connections for transmitting physiological data, results of the analysis by the microcontroller 12, and alerts and/or alarms to the patient, clinicians and caregivers regarding any abnormal conditions detected. Additionally, the memory in the microcontroller 13 stores software or algorithms with executable instructions and the microcontroller 13 can execute a set of instructions of the software or algorithms in association with establishing communication connections with various devices and networks using both wired and wireless connections.

As shown in FIG. 1, wireless communication connections established by the communication interface circuitry of microcontroller 13 include a Bluetooth connection 14, a cellular network connection 16, and a WiFi connection 17. The wireless communication connections allow for alerts and physiological data to be transmitted in real-time within a hospital wireless communications network (e.g., WiFi) as well as allow for alerts and physiological data to be transmitted in real-time to other devices (e.g., Bluetooth and cellular networks). For example, if the patient monitor (i.e., main module 7) detects a physiological event, an alert or alarm along with pertinent data can transmitted through the cellular network 16 to the clinician and/or health care facility. As another example, if the Bluetooth connection 14 or WIFI connection 17 are not available (e.g., out of transmission range or not operable), and a significant physiological event is detected, the microcontroller 13 can transmit the physiological event, and an alert along with pertinent data using the cellular network connection 16.

It is also contemplated by the disclosure of the present application that the communication connections established by the microcontroller 13 enable communications over other types of wireless networks using alternate hospital wireless communications such as wireless medical telemetry service (WMTS), which can operate at specified frequencies (e.g., 1.4 GHz). Other wireless communication connections can include wireless connections that operate in accordance with, but are not limited to, IEEE802.11 protocol, a Radio Frequency For Consumer Electronics (RF4CE) protocol, ZigBee protocol, and/or IEEE802.15.4 protocol.

The Bluetooth connection 14 can be used to provide the transfer of data to a nearby device (e.g. tablet) for review of data and/or changing of operational settings of the main module 7. The Bluetooth connection 14 also provides wireless communications between the main module 7 and wireless physiological sensors (e.g., ECG, SpO2). Wireless physiological sensors have the advantage of eliminating wires, which get tangled, disconnected, or fail. The microcontroller 13 of the main module 7 provides communication connection by direct wired (e.g., hard-wired) connections as well for transferring data using, for example, a USB connection 19 to a tablet, PC, or similar electronic device; or using, for example, a USB connection 20 to an external storage device or memory.

Additionally, the microcontroller 13 includes a connection to a graphical user interface (GUI) 21 for displaying information, physiological data, measured data, and/or alerts/alarms to the patient, or to clinicians and caregivers proximate to the main module 7. Although the main module 7 is described in FIG. 1 as having two microcontrollers 12, 13, it is contemplated by the disclosure of the present application that one microcontroller could be implemented to perform the functions of the two microcontrollers 12, 13.

The GUI 21 is, for example, a liquid crystal display (LCD), cathode ray tube (CRT), thin film transistor (TFT), light-emitting diode (LED), high definition (HD) or other similar display device with touch screen capabilities. The GUI is provided with means for inputting instructions or information directly to the main module 7.

As shown in FIG. 1, the main module 7 also includes a GPS 18 that can transmit to the clinician or caregiver the location of the patient. If it is determined by the microcontroller 13 that the patient is not within the vicinity of the hospital wireless communications system (e.g., based on input from the GPS 18), the pertinent physiological data (e.g., full disclosure and physiological signal measurements) can be recorded and stored in an on-board memory 22. Additionally, if the Bluetooth connection 14 or WIFI connection 17 are not available (e.g., out of transmission range or not operable), and a physiological event detected is not significant, then the microcontroller can stored the physiological data (e.g., full disclosure and physiological signal measurements) in the on-board memory 22 for later transmission when the Bluetooth connection or WIFI connection become available.

The on-board memory 22 is, for example, a random access memory (RAM), a memory buffer, a hard drive, a database, an erasable programmable read only memory (EPROM), an electrically erasable programmable read only memory (EEPROM), a read only memory (ROM), a flash memory, or a hard disk.

Power can be supplied to the main module 7 using a rechargeable battery 4 that can be detached allowing for replacement. The rechargeable battery 4 is, for example, a rechargeable lithium-ion battery. Additionally, a small built-in back-up battery 6 (or super capacitor) is provided for continuous power to main module 7 during battery replacement. A power supply regulation circuit 5 is provided between the rechargeable battery 4 and small back-up battery 6 to control which of batteries 4, 6 provide power to the main module 7. The main module 7 also includes a patient ground connection 2 for providing a reference when acquiring the ECG signals. The patient ground connection 2 can be used as a ground for single ended unipolar input amplifiers (e.g., precordial leads), or as a ground for bipolar input amplifiers (e.g., limb leads).

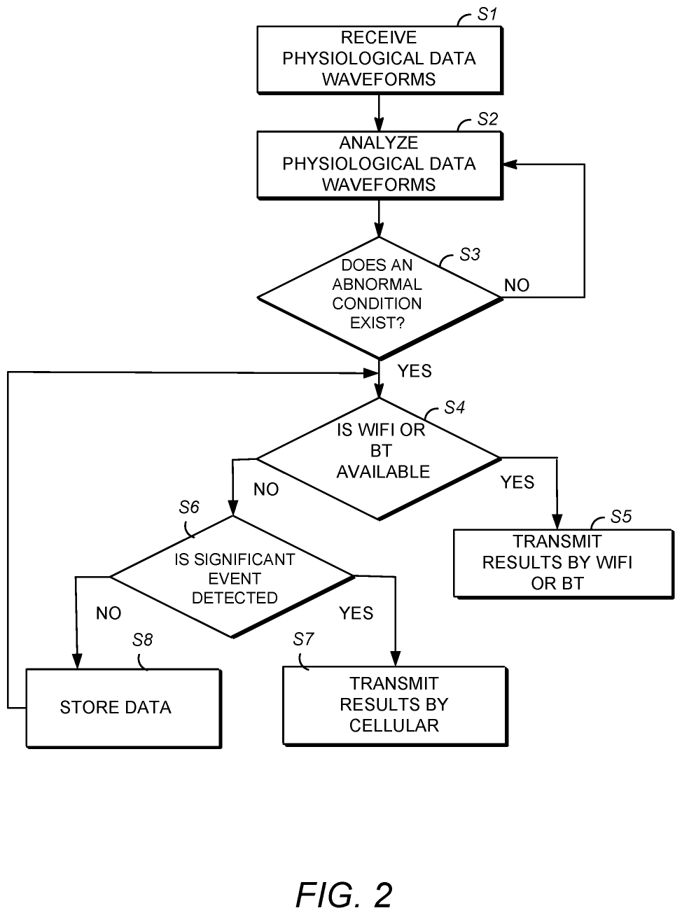

FIG. 2 is an exemplary algorithm executed by the microcontrollers of the main module according to an embodiment of the present disclosure.

In step S1, the microcontroller 12 of the main module 7 receives the digital data waveforms from the ECG data acquisition circuit 9, the SpO2 data acquisition circuit 8, and external physiological parameter interface 11. The memory of the microcontroller 12 has stored in advance digital waveform characteristics and threshold levels indicative of abnormal conditions of the patient. In step S2, the microcontroller 12 analyzes the received digital data waveforms using the stored digital waveform characteristics and threshold levels, and identifies any abnormal conditions by comparing the stored digital waveform characteristics and threshold levels with the received digital data waveforms. In step S3, if it is determined that no abnormal condition exists, the microcontroller 12 continues to analyze the received digital data waveforms that are received, as in step S2. However, in step S3, if it is determined that any abnormal condition exists, then the microcontroller 12 transmits the results of the analysis to the microcontroller 13 of the main module 7 by an electrical connection between the microcontrollers 12, 13.

In step 4, the microcontroller 13 determines if the WIFI connection 17 or the Bluetooth connection 14 is available for transmissions. For example, the microcontroller 13 may determine that the main module 7 is not within transmission range for using the WIFI connection 17 or the Bluetooth connection 14, or determine that the WIFI connection 17 or the Bluetooth connection 14 is not operable. If it is determined by the microcontroller 13 that the WIFI connection 17 or the Bluetooth connection 14 is available, then in step S5 the microcontroller 13 transmits the physiological data and alerts along with other pertinent data using the WIFI connection 17 or the Bluetooth connection 14.

However, in step S4, if it is determined that the WIFI connection 17 or the Bluetooth connection 14 is not available for transmissions, then in step S6 the microcontroller 13 determines if a significant physiological event has been detected (e.g., a significant physiological event requiring immediate attention by a physician or caregiver). If it is determined that a significant physiological event has been detected, then in step S7 the microcontroller 13 transmits the physiological data and alerts along with other pertinent data using the cellular connection 16. However, if it is determined that no significant physiological event has been detected, then in step S8 the microcontroller 13 stores the physiological data in the on-board memory 22 for later transmission when the WIFI connection 17 or the Bluetooth connection 14 become available, as determined in steps S4-S5.

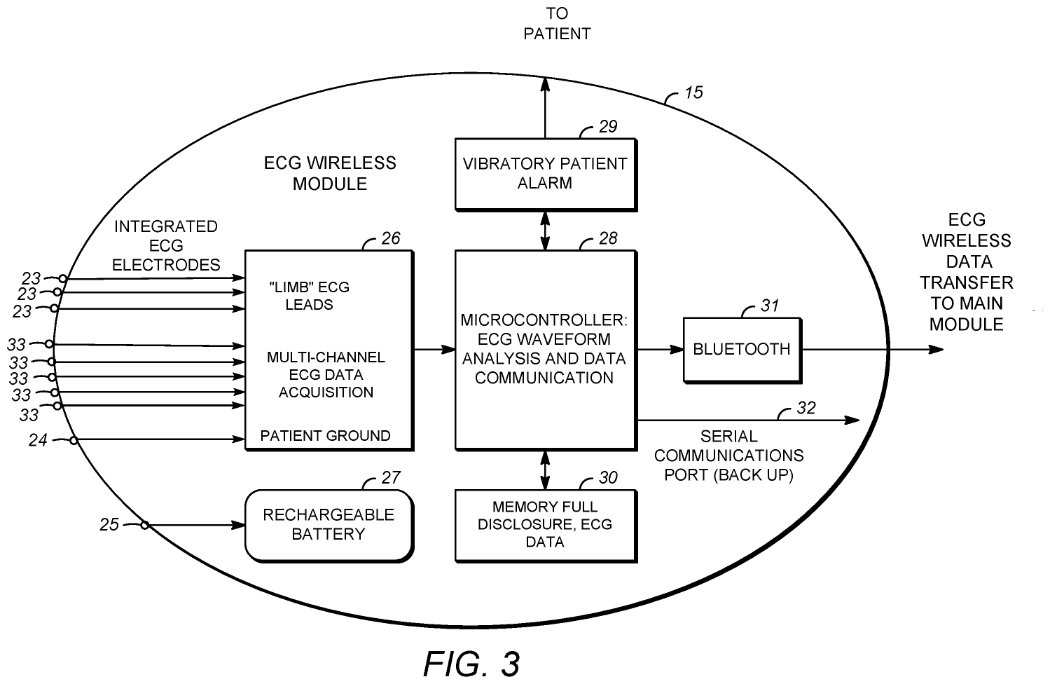

FIG. 3 is a block diagram showing a wireless electrocardiogram (ECG) module according to an embodiment of the present disclosure. The wireless ECG module 15 can have two configurations for physiological data acquisition. During continuous monitoring, the wireless ECG module 15 is connected to a minimal set (e.g., 3) of ECG electrodes 23 that provide data signals related to an electrocardiogram (ECG), similar to 3 channel limb leads known in the art. In an alternative embodiment, additional ECG electrodes can be added. For example, a fourth ECG electrode (not shown) can be added, which can be provided as a ground reference for the data acquisition circuits 26. Additionally, when an acute recording of a 12 lead ECG configuration is required, an additional set of electrodes (e.g., 5 or more) can be added and placed in precordial locations. For example, a precordial array of electrodes can be connected to the wireless ECG module 15 using electrical connections 33 to the data acquisition circuitry 26 (e.g., each electrode having a separate connection 33 to the data acquisition circuitry 26). From the above example, a 12 lead ECG can be derived from the ECG data signals of the 9 electrodes.

As shown in FIG. 3, the ECG electrodes 23 transmit ECG data signals to the data acquisition circuit 26 of the wireless ECG module 15. The ECG electrodes are integrated with the wireless ECG module and transmit ECG data signals by an electrical connection therebetween. The data signals from the ECG electrodes 23 are, for example, analog signals. The data signals from the ECG electrodes 23 are input to an ECG data acquisition circuit 26, which is similar to the data acquisition circuit 9 of the main module 7. That is, the ECG data acquisition circuit 26 includes amplifying circuitry, filtering circuitry, and A/D circuitry that convert the analog signals to digital signals using amplification, filtering, and A/D conversion methods known in the art.

The processing of the ECG data signals by the ECG data acquisition circuit 26 produces digital data waveforms, which are passed to a microcontroller 28 by electrical connection therebetween. The microcontroller 28 analyzes the digital waveforms to identify certain digital waveform characteristics and threshold levels indicative of abnormal conditions of the patient. The microcontroller 28 is, for example, a processor, a field programmable gate array (FPGA), an application specific integrated circuit (ASIC), a digital signal processor (DSP), or other similar processing device. The microcontroller 28 also includes a memory. The memory is, for example, a random access memory (RAM), a memory buffer, a hard drive, a database, an erasable programmable read only memory (EPROM), an electrically erasable programmable read only memory (EEPROM), a read only memory (ROM), a flash memory, or a hard disk.

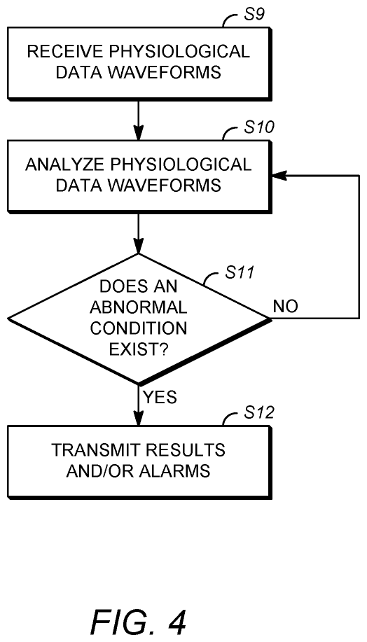

The memory stores software or algorithms with executable instructions and the microcontroller 28 can execute a set of instructions of the software or algorithms in association with executing an operation of analyzing the digital data waveforms related to the data signals of the ECG electrodes 23 to identify abnormal conditions of the patient. FIG. 4 is an exemplary algorithm executed by a microcontroller 28 of the wireless ECG module 15. In step S9, the microcontroller 28 of the wireless ECG module 15 receives the digital data waveforms from the ECG data acquisition circuit 26. The memory of the microcontroller 28 has stored in advance digital waveform characteristics and threshold levels indicative of abnormal conditions of the patient. In step S10, the microcontroller 28 analyzes the received digital data waveforms using the stored digital waveform characteristics and threshold levels, and identifies any abnormal conditions. The microcontroller 28 can identify any abnormal cardiac conditions (e.g. arrhythmias, or ST segment measurements indicative of ischemia or myocardial infarction).

In step S11, if it is determined that no abnormal condition exists, the microcontroller 28 continues to analyze the physiological data waveforms received, as in step S10. However, in step S11, if it is determined that any abnormal condition exists, then in step S12 the microcontroller 28 transmits the results to the patient in the way of an alert or alarm, and/or transmits the results to the main module 7 via a wireless Bluetooth connection 31.

The memory in the microcontroller 28 stores software or algorithms with executable instructions and the microcontroller 28 can execute a set of instructions of the software or algorithms in association with establishing communication connections with various devices and networks using the wireless communication interface circuitry of the microcontroller 28.

Referring again to FIG. 3, wireless communication connections established by the wireless communication interface circuitry of microcontroller 28 include a Bluetooth connection 31 to the main module 7. The Bluetooth connection 31 enables the microcontroller to transmit alerts and physiological data to the main module 7 in real-time. It is also contemplated by the disclosure of the present application that the communication connections established by the microcontroller 28 enable communications over other types of wireless networks such as wireless connections that operate in accordance with, but is not limited to, IEEE802.11 protocol, a Radio Frequency For Consumer Electronics (RF4CE) protocol, ZigBee protocol, and/or IEEE802.15.4 protocol. As a backup to the wireless connection, alerts and physiological data can be transmitted to the main module 7 in real-time using a serial connection 32.

The microcontroller 28 can also transmit a signal to an internal alarm 29 (i.e., if an abnormal condition is detected) using, for example, a vibratory response to the patient's skin to directly alert the patient. Additionally, pertinent physiological data (e.g., full disclosure and physiological signal measurements) can be stored in on-board memory 30 electrically connected to the microcontroller 28. The on-board memory 30 is, for example, a random access memory (RAM), a memory buffer, a hard drive, a database, an erasable programmable read only memory (EPROM), an electrically erasable programmable read only memory (EEPROM), a read only memory (ROM), a flash memory, or a hard disk. As shown in FIG. 3, power can be supplied to the wireless ECG module 15 by a rechargeable battery 27 that can be recharged via a power connection 25 to the wireless ECG module 15. The rechargeable battery 27 is, for example, a rechargeable lithium-ion battery that can be detached allowing for replacement. The wireless ECG module 15 also includes a patient ground connection 24 for providing as a reference when acquiring the ECG signals. The patient ground connection 24 can be used as a ground for single ended unipolar input amplifiers (e.g., precordial leads), or as a ground for bipolar input amplifiers (e.g., limb leads).

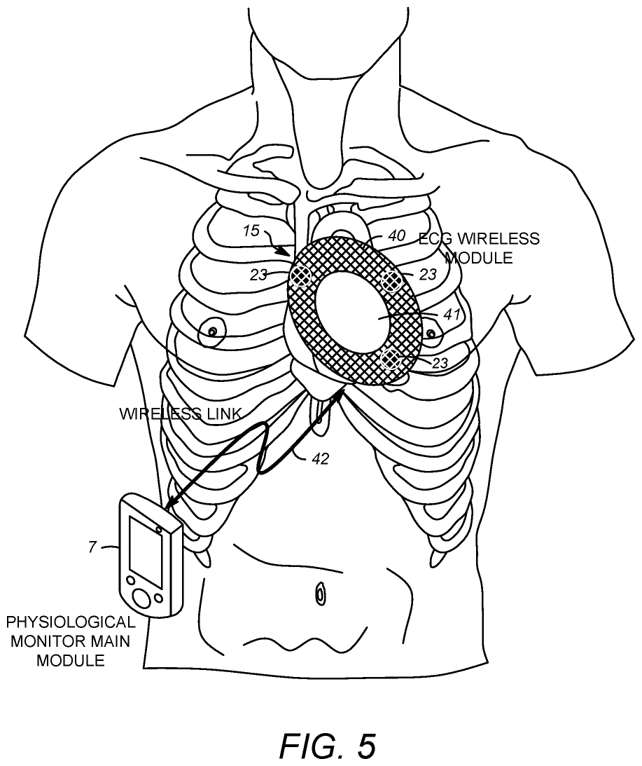

FIG. 5 is a diagram of a physiological monitoring system according to an embodiment of the present disclosure.

As shown in FIG. 5, the physiological monitoring system includes the main module 7 and the wireless ECG module 15 that communicate with each other via a wireless communication link 42. In a preferred embodiment, the wireless communication link 42 established between main module 7 and the wireless ECG module 15 is implemented in accordance with a Bluetooth protocol. The wireless communication link 42 enables the wireless ECG module 15 to transmit alerts and physiological data to the main module 7 in real-time. It is also contemplated by the disclosure of the present application that the wireless communication link 42 established by the wireless ECG module 15 and the main controller 7 is in accordance with other wireless protocols such as, but not limited to, IEEE802.11 protocol, a Radio Frequency For Consumer Electronics (RF4CE) protocol, ZigBee protocol, and/or IEEE802.15.4 protocol. As a backup to the wireless communication link 42, alerts and physiological data is transmitted to the main module 7 in real-time using, for example, a serial connection 32 of the wireless ECG module 15.

As shown in FIG. 5, the main module 7 includes a user interface that provides a means for inputting instructions or information directly to the main module 7. The user interface includes a display screen that is, for example, a liquid crystal display (LCD), cathode ray tube (CRT), thin film transistor (TFT), light-emitting diode (LED), high definition (HD) or other similar display device with touch screen capabilities. The user interface of the main module 7 also provides controls to optimize the viewing of data, which include, but are not limited to, keys, buttons, knobs, touch screen or other similar input devices that can be used to input instructions to the main module 7.

As shown in FIG. 5, the main module 7 is worn by the patient (e.g., on the hip) using a mechanical device or mechanism such as, but not limited to, a clip or strap attached to a surface of the main module 7. However, the main module 7 is detachable and can be removed and held by the patient or set down at a location proximate to the patient. For example, the main module 7 can be placed in a nearby location when the patient is stationary or sleeping (e.g., on a night table).

The wireless ECG module 15 includes a data acquisition module 41 composed of a flexible polymer material with embedded circuitry and an electrode patch 40. The embedded circuitry of the data acquisition module 41 includes, for example, the data acquisition circuit 26, microcontroller 28, on-board memory 30, the rechargeable battery 27, and patient alarm 29, as described with reference to FIG. 3. The electrode patch 40 of the wireless ECG module 15 is composed of, for example, a breathable porous material integrated with a minimal set (e.g., 3) of ECG electrodes 23. In an alternative embodiment, additional ECG electrodes can be added. Additionally, the electrode patch 40 can also be composed of silicon, polymer, foam, cloth, or similar material.

The electrode patch 40 of the wireless ECG module 15 is attached to the chest of the patient using, for example, a biocompatible adhesive or an adhesive surface on the bottom surface of the electrode patch 40 facing the patient's skin. However, the electrode patch 40 can be made from a material that is self-adhesive. As shown in FIG. 5, the wireless ECG module 15 is located over the heart (e.g., left side of chest) with the electrode patch 40 including the ECG electrodes 23 (e.g., 3) located in a similar orientation as the typical 3 lead (LA, RA, LL) limb lead configuration known in the art. The orientation of the electrodes 23 can also be placed in alternative positions on the patient's chest depending on the patient's anatomy or the area of the heart of diagnostic interest. Once attached to the patient, the electrical signals from the electrodes 23 are received by the data acquisition module 41 using flexible wire interconnections in the wireless ECG module 15 that connect the electrodes to the embedded circuitry of the data acquisition module 41.

The ECG data signals from the electrodes 23 are received by the data acquisition module 41, and processed by the embedded circuitry as described previously with reference to FIGS. 3 and 4. For example, the data signals from the ECG electrodes 23 are input to an ECG data acquisition circuit 26, which includes amplifying circuitry, filtering circuitry, and A/D circuitry that convert the analog signals to digital signals using amplification, filtering, and A/D conversion methods known in the art.

The processing of the ECG data signals by the ECG data acquisition circuit 26 produces digital data waveforms that are passed to a microcontroller 28, which analyzes the digital waveforms to identify certain digital waveform characteristics and threshold levels indicative of abnormal conditions of the patient. The microcontroller 28 can identify any abnormal cardiac conditions (e.g. arrhythmias, or ST segment measurements indicative of ischemia or myocardial infarction).

If it is determined that any abnormal condition exists, then the microcontroller 28 transmits the results to the patient in the way of an alert or alarm, and/or transmits the results to the main module 7 via the wireless communication link 42. When transmitting an alert or alarm to the patient, the microcontroller 28 transmits a signal to the internal alarm 29 using, for example, a vibratory response directly to the patient's skin. When the results are transmitted to the main module 7 via the wireless communication link 42, the microcontroller 13 of the main module 7 can establish communication connections using both wired and wireless connections for transmitting physiological data, results, and alerts and/or alarms to the patient, clinicians, and caregivers regarding any abnormal conditions detected as well as store the physiological data in the on-board memory 22, as described previously with reference to FIGS. 1 and 2.

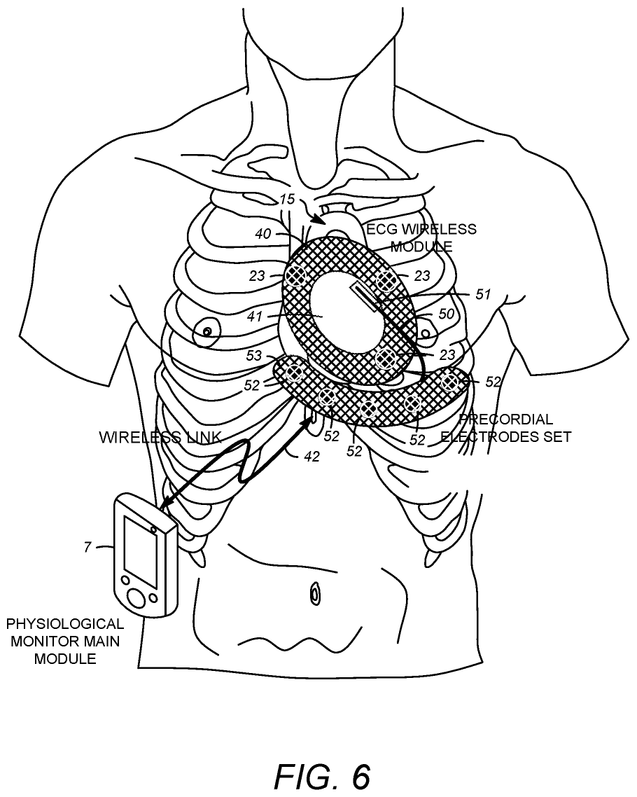

FIG. 6 is a diagram of a physiological monitoring system including a precordial electrode array connected to the wireless ECG module according to an embodiment of the present disclosure.

The physiological monitoring system of FIG. 6 differs from FIG. 5 in that in the physiological monitoring system of FIG. 6 includes a precordial electrode array (or set) 53. The precordial electrode array 53 can be placed over the precordial locations of the right and left chest proximate to the ECG wireless module 15. It is known in the art that the precordial locations are the best locations to detect the heart's electrical activity associated with the septal surface, the anterior wall of the right and left ventricles, and lateral wall of the left ventricle. The precordial electrode array 53 is composed of a breathable porous material integrated with the electrodes 52 (e.g., 5 or more electrodes). However, the precordial electrode array 53 can also be composed of silicon, polymer, foam, cloth, or similar material. The precordial electrode array 53 is attached to the patient using, for example, a biocompatible adhesive or an adhesive surface on the bottom surface of the precordial electrode array 53 facing the patient's skin. However, the precordial electrode array 53 can be made from a material that is self-adhesive.

As shown in FIG. 6, the precordial electrode array 53 is connected to the wireless ECG module 15 through, for example, an external connection port (e.g., ECG cable port) in the data acquisition module 41. The precordial electrode set 53 includes a cable 50 that terminates with an in-line connector 51. The in-line connector 51 is received in the external connection port (e.g., ECG cable port) of the data acquisition module 41, which establishes an electrical connection with the embedded circuitry (e.g., connections 33 to the data acquisition circuit 26), thereby also establishing a connection between the precordial electrode array 53 and the wireless ECG module 15. This configuration provides the capability of obtaining additional ECG data for a diagnostic data similar to the 12 lead recording without having to remove the primary electrodes (e.g., 3) of the wireless ECG module 15. A ground reference for the precordial electrode array 53 can be a remote electrode located remote from the precordial set (not shown). Alternatively, the ground reference can be derived from the 3 lead set received through the wireless communication link 42, and converted back to an analog signal. Once the additional 12 lead ECG data is obtained, the precordial electrode array 53 can be disconnected after it is determined that the additional diagnostic ECG data is no longer required. Thus, the 3 lead set does not have to be moved or removed during extended 12 lead ECG recording.

The cable 50 between the precordial electrode set 53 and the wireless ECG module 15 is, for example, an electrical cable or other similar interface cable. Once attached, the electrical signals from the electrodes 52 of the precordial electrode array 53 are received by the data acquisition module 41 of the wireless ECG module 15, and the data signals are processed by the embedded circuitry as described previously with reference to FIGS. 3 and 4.

For example, the data signals from the electrodes 52 are input to an ECG data acquisition circuit 26, which includes amplifying circuitry, filtering circuitry, and A/D circuitry that convert the analog signals to digital signals using amplification, filtering, and A/D conversion methods known in the art. The processing of the ECG data signals by the ECG data acquisition circuit 26 produces digital data waveforms that are passed to a microcontroller 28, which analyzes the digital waveforms to identify certain digital waveform characteristics and threshold levels indicative of abnormal conditions of the patient. The microcontroller 28 can identify any abnormal cardiac conditions (e.g. arrhythmias, or ST segment measurements indicative of ischemia or myocardial infarction).

If it is determined that any abnormal condition exists, then the microcontroller 28 transmits the results to the patient in the way of an alert or alarm, and/or transmits the results to the main module 7 via the wireless communication link 42. When transmitting an alert or alarm to the patient, the microcontroller 28 transmits a signal to the internal alarm 29 using, for example, a vibratory response directly to the patient's skin. When the results are transmitted to the main module 7 via the wireless communication link 42, the microcontroller 13 of the main module 7 can establish communication connections using both wired and wireless connections for transmitting physiological data, results, and alerts and/or alarms to the patient, clinicians, and caregivers regarding any abnormal conditions detected as well as store the physiological data in the on-board memory 22, as described previously with reference to FIGS. 1 and 2.

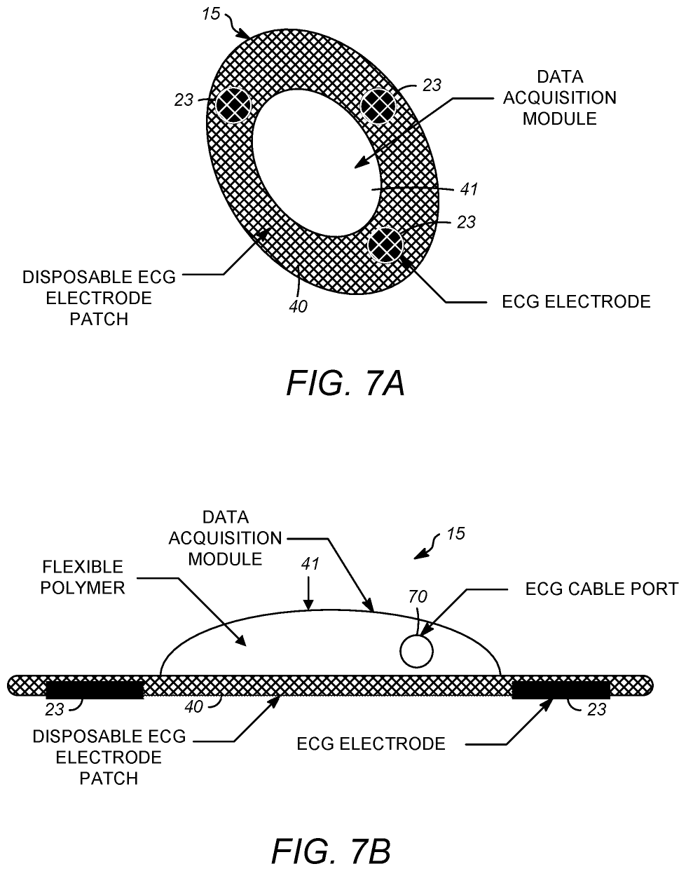

FIG. 7A is a top view of the wireless ECG module and FIG. 7B is a side view of the wireless ECG module according to an embodiment of the present disclosure.

As shown in FIG. 7A, the wireless ECG module 15 includes a data acquisition module 41 with embedded circuitry and an electrode patch 40. The embedded circuitry of the data acquisition module 41 includes, for example, the data acquisition circuit 26, microcontroller 28, on-board memory 30, the rechargeable battery 27, and patient alarm 29, as described with reference to FIG. 3. The data acquisition module 41 is composed of, for example, a flexible polymer or other similar material.

The electrode patch 40 of the wireless ECG module 15 is composed of, a breathable porous material integrated with a minimal set (e.g., 3) of ECG electrodes 23, similar to 3 channel limb leads known in the art. However, the electrode patch can also be composed of silicon, polymer, foam, cloth, or similar material.

In this embodiment, the ECG data acquisition module 41 can be a reusable device and detachably connected to the electrode patch 40, whereas the electrode patch 40 (including the electrodes) is disposable. The ECG electrode patch 40 is composed of a material that is a flexible porous structure to allow the patient's skin to “breathe.” The ECG data acquisition module 41 is composed of a flexible material such as a polymer that is resistant to water ingress, which allows the patient to take a shower or bath while wearing the wireless ECG module 15. The polymer can also be flexible to allow the wireless ECG module 15 to conform to the patient's body. Once the ECG data acquisition module 41 is attached to a new electrode patch 40, the three (3) electrodes are connected underneath the ECG electrode patch by flexible circuitry to the data acquisition module 41 for detecting the ECG voltage signals.

FIG. 7B illustrates a side view of the wireless ECG wireless module 15 with the ECG data acquisition module 41 attached to the ECG electrode patch 40. As shown in FIG. 7B, the electrodes 23 are located on the outer periphery of the electrode patch 40 with a bottom surface of the electrodes configured to come into contact with the patient's skin. In this embodiment, the ECG data acquisition module 41 is a reusable device and detachably connected to the electrode patch 40, whereas the electrode patch 40 (including the electrodes) is disposable and composed of a material that is a flexible porous structure to allow the patient's skin to “breathe.” The ECG data acquisition module 41 is composed of a flexible material (e.g., silicone or polymer) that provides protection for the electrical interconnects between the data acquisition module 41 and the electrode patch 40 against environmental hazards such as moisture.

When the patient is within a higher acuity level of physiological monitoring, there can still be a requirement to obtain a 12 lead ECG recording at certain times throughout the course of a day for diagnostic purposes. It is also preferable that the clinicians do not have to remove the electrodes for the 3 lead configuration, to attach electrodes for a separate 12 lead ECG recorder. Under such conditions, a separate precordial ECG electrode array 53 can be attached directly to the patient and the wireless ECG module 15 using the cable port 70 in the data acquisition module 41 for providing additional ECG recordings in precordial locations during the higher acuity monitoring. The precordial electrode set 53 includes a cable 50 that terminates with an in-line connector 51. The in-line connector 51 is received in the ECG cable port 70 of the data acquisition module 41, which establishes an electrical connection with the embedded circuitry (e.g., connections 33 to the data acquisition circuit 26), thereby also establishing a connection between the precordial electrode array 53 and the wireless ECG module 15.

The ECG electrode array 53 can also be used during cardiac rehabilitation activities to monitor the 12 lead ECG signal. Once the requirement for obtaining the precordial ECG waveforms is no longer required, the precordial ECG electrode array 53 can be disconnected from the wireless ECG module 15 by disconnecting the in-line connector 51 of the cable 50 from the ECG cable port 70 of the wireless ECG module. The precordial electrode array 53 can be removed from the patient leaving the primary 3 lead ECG patch 40 of the wireless ECG module 15 attached for additional continuous 24 hour (or greater) monitoring.

In another embodiment of the ECG electrode patch 40 of the ECG wireless module 15, the ECG electrode patch 40 can include additional electrodes (e.g. 5 or more) embedded within a flexible polymer insulation for detecting the ECG voltage signal as a precordial ECG electrode array.

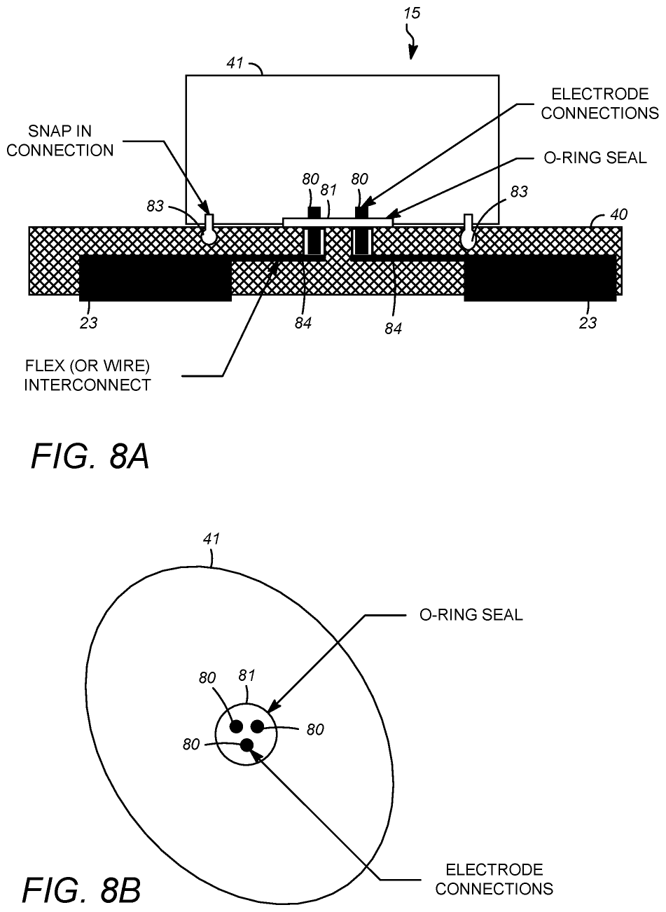

FIG. 8A is side view of the connection between the data acquisition module and the electrode patch of the ECG module according to an embodiment of the present disclosure.

As shown in FIG. 8A, the connections between the data acquisition module 41 and the electrode patch 40 includes mechanical connections 83, electrical connections 80, 84, and an O-ring seal 81 to protect against water ingress, which allows the patient to shower or take a bath while wearing the wireless ECG module 15. The mechanical connections are, for example, snap plug mechanisms 83 that snap into the surface of the electrode patch 40, thereby detachably securing the bottom surface of the data acquisition module 41 to the top surface of the electrode patch 40. The electrical connections include, for example, male electrical connectors 80 (e.g., 3) that establish an electrical connection with the electrodes 23 of the electrode patch 40 by coming in contact with the wires or flexible circuit 84 embedded in the electrode patch 40. The electrical connections 80, 84 allow for ECG signals from the ECG electrodes 23 to be transmitted to the ECG data acquisition module 41. Although FIG. 8A shows the use of male connectors 80, it is also contemplated by the disclosure of the present application that the connectors used for establishing connections between the data acquisition module 41 and the electrode patch 40 are female connectors or a combination of male and female connectors.

In order to protect against water ingress to the electrical connection between the data acquisition module 41 and the electrode patch 40, there is an O-ring seal 81 around the electrical connectors 80 and positioned between the bottom surface of the data acquisition module 41 and the top surface of the ECG electrode patch 40.

FIG. 8B is a bottom view of the electrode connections of the data acquisition module according to an embodiment of the present disclosure.

As shown in FIG. 8B, the bottom surface of the data acquisition module 41 includes an O-ring seal 81 that is concentric and surrounding the electrical connectors 80 (e.g., 3 connectors) in order to provide a water tight seal, which protects the integrity of the electrical connection between the data acquisition module 41 and the electrode patch 40. The water tight seal created by the O-ring seal 81 also allows the patient to take a shower or bath while wearing the wireless ECG module 15.

The electrical connectors 80 are, for example, male electrical connectors (e.g., 3) that establish an electrical connection with the electrodes 23 of the electrode patch 40 coming in contact with the wires or flexible circuit 84 embedded in the electrode patch 40. It is contemplated by the disclosure of the present application that the connectors used for establishing connections between the data acquisition module 41 and the electrode patch 40 can be male or female connectors or a combination of male and female connectors.

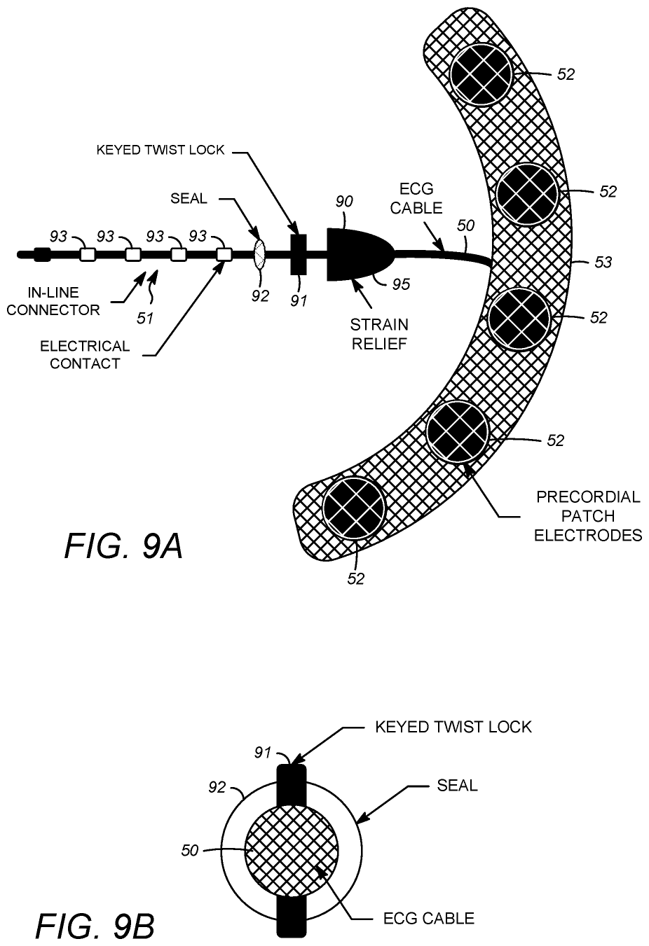

FIG. 9A is top view of a precordial electrode array with cable and in-line connector according to an embodiment of the present disclosure.

A separate precordial ECG electrode array 53 can be attached directly to the patient and the wireless ECG module 15 using the cable port 70 in the data acquisition module 41 for providing additional ECG recordings in precordial locations during the higher acuity monitoring. The precordial electrode array 53 is attached to the chest of the patient using, for example, a biocompatible adhesive or an adhesive surface on the bottom surface of the precordial electrode array 53 facing the patient's skin. However, the precordial electrode array 53 can be made from a material that is self-adhesive. The precordial electrode array 53 is connected to the primary set of electrodes (e.g., 3 electrodes) of the wireless ECG module 15 through an external connection port (e.g., ECG cable port) in the data acquisition module 41.

The precordial electrode array 53 is composed of a breathable porous material integrated with the electrodes 52 (e.g., 5 or more electrodes). However, the precordial electrode array 53 can also be composed of silicon, polymer, foam, cloth, or similar material. As shown in FIG. 9A, the precordial electrode array 53 includes a cable 50 that terminates with an in-line connector 51. The in-line connector 51 includes a series of electrical contacts 93 that are received in the external connection port (e.g., ECG cable port 70) of the data acquisition module 41 and establish an electrical connection with the embedded circuitry (e.g., connections 33 to the data acquisition circuit 26) and thereby a connection between the precordial electrode array 53 and the wireless ECG module 15. This configuration provides the capability of obtaining additional ECG data as diagnostic data similar to the 12 lead recording without having to remove the primary electrodes (e.g., 3) of the wireless ECG module 15.

The in-line connector 51 also includes a seal 92 that is concentric around the ECG cable 50 for providing a water tight seal, thereby protecting the integrity of the electrical connection between the data acquisition module 41 and the precordial electrode array 53 when the in-line connector is received in the external connection port (e.g., ECG cable port 70) of the data acquisition module 41. Having the seal 91 integrated within the ECG cable 50 rather than having an O-ring within the connector cavity can improve the reliability of the seal by avoiding a multi-use configuration.

The in-line connector 51, once establishing a connection between the data acquisition module 41 and the precordial electrode array 53, is held securely in place using a lock mechanism such as a keyed twist lock 91. When the keyed twist lock 91 is inserted in the external connection port of the data acquisition module 41, the keyed twist lock 91 is twisted (e.g., 90° in the clockwise direction) and aligned within a grooved slot (e.g., in the external connection port). The In-line connector 51 is guided into place in the external connection port of the data acquisition module 41 by holding the strain relief portion 90 of the in-line connector 51, which prevents damage to the precordial electrode array 53 and the cable 50. The strain relief portion 90 is integrated into the ECG cable 50, which can also provide additional protection from water ingress.

The ECG electrode array 53 can be used during cardiac rehabilitation activities to monitor the 12 lead ECG signal. Once the requirement for obtaining the precordial ECG waveforms is no longer required, the precordial ECG electrode array 53 can be disconnected from the wireless ECG module 15 by disconnecting the in-line connector 51 of the cable 50 from the ECG cable port 70 of the wireless ECG module 15. The precordial electrode array 53 can be removed from the patient leaving the primary 3 lead ECG patch 40 of the wireless ECG module 15 attached for additional continuous 24 hour (or greater) monitoring.

FIG. 9B is a cross-sectional view of the keyed twist lock of the ECG cable for the precordial electrode array according to an embodiment of the present disclosure.

The in-line connector 51, once in place and establishing a connection between the data acquisition module 41 and the precordial electrode array 53, is held securely in place using an integrated keyed twist lock 91.

As shown in FIG. 9B, the keyed twist lock 91 is integrated with the cable 50, similar to the seal 92. The key twist lock 91 is inserted into a grooved slot of the external connection port (e.g., ECG cable port 70) of the data acquisition module 41 and twisted, for example, 90° in a clockwise direction and aligned within a grooved slot, thereby securing the connection between the in-line connector 51 and the data acquisition module 41. Once the requirement for obtaining the precordial ECG waveforms is no longer required, the precordial ECG electrode array 53 can be disconnected from the wireless ECG module 15 by again twisting the key twist lock 91 inserted into a grooved slot of the external connection port by, for example, 90° in a counter clockwise direction, thereby releasing the connection between the in-line connector 51 and the data acquisition module 41.

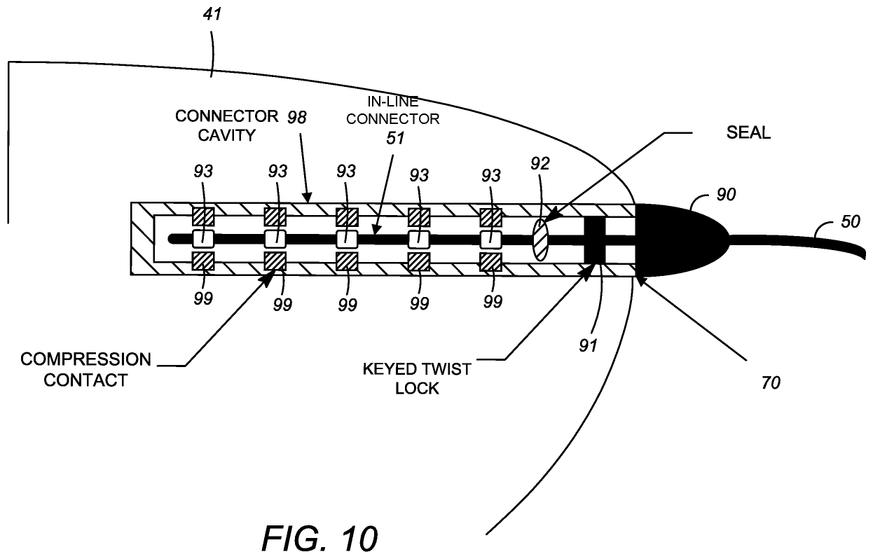

FIG. 10 is a side view of the connection between the data acquisition module and the precordial electrode array according to an embodiment of the present disclosure.

The precordial electrode array 53 includes a cable 50 that terminates with an in-line connector 51. As shown in FIG. 10, the in-line connector 51 includes a series of electrical contacts 93 that are received in the ECG cable port 70 of the data acquisition module 41. The ECG cable port 70 includes a connector cavity 98 having a series of compression contacts 99 that align with the series of electrical contacts 93 when the in-line connector 51 is fully inserted into the connector cavity 98 of the data acquisition module 41. When the in-line connector 51 is fully inserted into the connector cavity 98 of the data acquisition module 41, the in-line connector 51 establishes an electrical connection with the embedded circuitry (e.g., connections 33 to the data acquisition circuit 26) of the data acquisition module 41, which in turn establishes a connection between the precordial electrode array 53 and the wireless ECG module 15. This configuration provides the capability of obtaining additional ECG data for diagnostic data similar to the 12 lead recording without having to remove the primary electrodes (e.g., 3) of the wireless ECG module 15.

The in-line connector 51 also includes an integrated seal 92 that is concentric around the ECG cable 50 for providing a water tight seal, thereby protecting the integrity of the electrical connection between the data acquisition module 41 and the precordial electrode array 53 when the in-line connector 51 is received in the ECG cable port 70 of the data acquisition module 41. Having the seal 92 integrated within the ECG cable 50 rather than having an O-ring within the connector cavity can improve the reliability of the seal by avoiding a multi-use configuration.

The in-line connector 51, once establishing a connection between the data acquisition module 41 and the precordial electrode array 53, is held securely in place using a lock mechanism such as a keyed twist lock 91, which when inserted in the ECG cable port 70 of the data acquisition module 41 is twisted (e.g., 90°) and aligned within a grooved slot. The In-line connector 51 is guided into ECG cable port 70 and the connector cavity 98 by holding the strain relief portion 90 of the in-line connector 51, which prevents damage to the precordial electrode array 53 and the cable 50. The strain relief portion 90 is integrated into the ECG cable 50, which can also provide additional protection from water ingress.

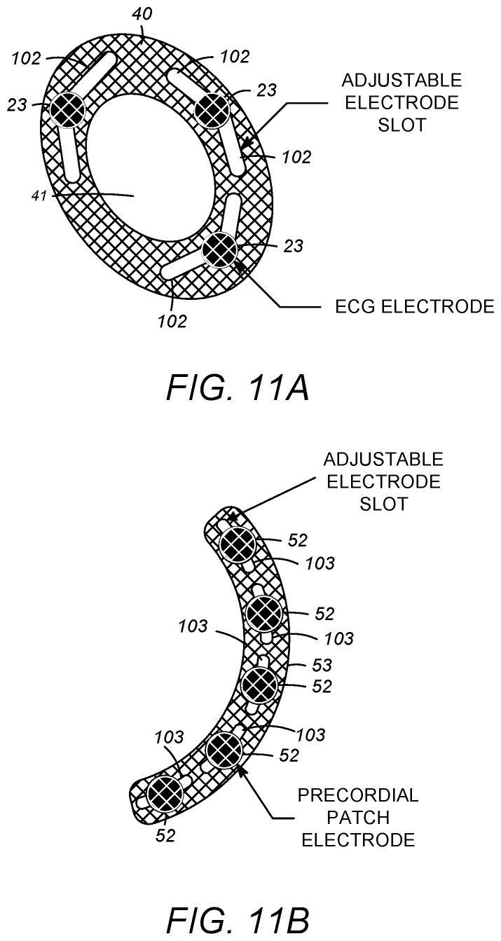

FIGS. 11A and 11B are top views respectively of the adjustable electrodes on the ECG module and the precordial array.

Each patient has a different anatomy (e.g., body size and orientation of heart) and may require monitoring for different cardiac conditions. Therefore, there may be a requirement to adjust the location of each ECG electrode to be application specific for each patient. As shown in FIG. 11A, this embodiment of the wireless ECG module 15 includes a data acquisition module 41 composed of a flexible polymer material with embedded circuitry and an electrode patch 40. The embedded circuitry of the data acquisition module 41 includes, for example, the data acquisition circuit 26, microcontroller 28, on-board memory 30, the rechargeable battery 27, and patient alarm 29, as described with reference to FIG. 3.

The electrode patch 40 of the wireless ECG module 15 is composed of, for example, a breathable porous material integrated with a minimal set (e.g., 3) of ECG electrodes 23. In an alternative embodiment, additional ECG electrodes can be added. Additionally, the electrode patch 40 can also be composed of silicon, polymer, foam, cloth, or similar material. As shown in FIG. 11A, the electrode patch 40 includes adjustable electrodes slots 102. The location of each ECG electrodes 23 can be adjusted within each of electrode slots 102 in the electrode patch 40. For example, one potential method to adjust each ECG electrode 23 would be to provide an adjustment to each ECG electrode 23 within its corresponding adjustable electrode slot 102 and relying on the compression of the flexible polymer of the electrode patch 40 around the body of the ECG electrodes 23 or an electrode adhesive to maintain stability.

As shown in FIG. 11B, this embodiment of the precordial electrode array 53 includes adjustable electrode slots 103. The precordial electrode array 53 is composed of a breathable porous material integrated with the electrodes 52 (e.g., 5 or more electrodes). However, the precordial electrode array 53 can also be composed of silicon, polymer, foam, cloth, or similar material. The location of each ECG electrode 52 can be adjusted within its corresponding electrode slot 103 in the precordial electrode array 53. For example, each ECG electrode 52 can be adjusted within its corresponding adjustable electrode slot 103 by relying on the compression of the flexible polymer of the around the body of the ECG electrodes 53 or an electrode adhesive to maintain stability.

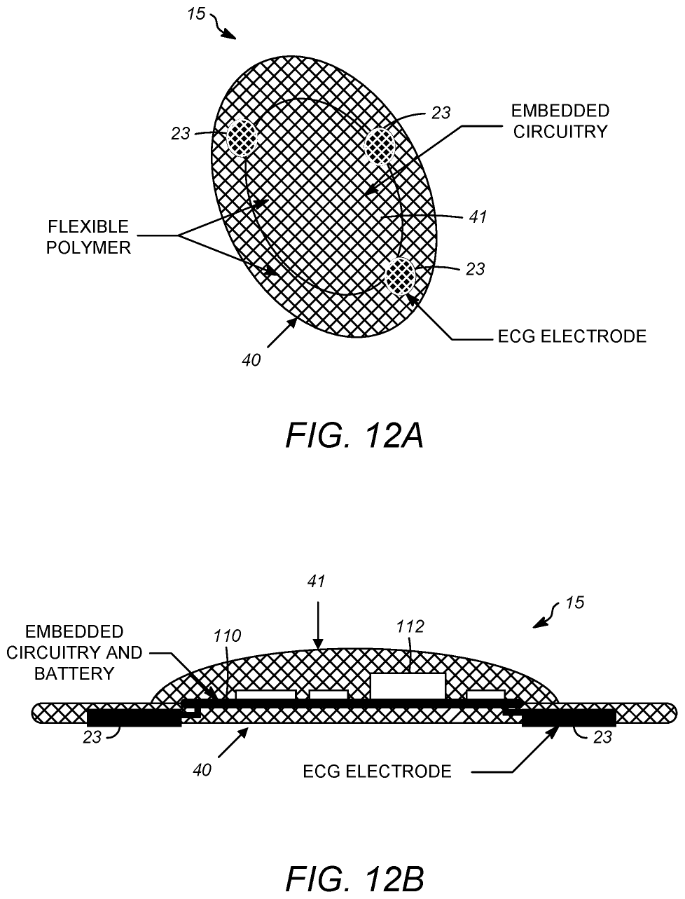

FIGS. 12A and 12B show an embodiment of the present disclosure in which the ECG data acquisition module 41 is integrated with the electrode patch 40 such that the entire ECG wireless device 15 is disposable. The wireless ECG module 15 is composed of, for example, a breathable porous material integrated with a minimal set (e.g., 3) of ECG electrodes 23. In an alternative embodiment, additional ECG electrodes can be added. Additionally, the wireless ECG module 15 also be composed of silicon, polymer, foam, cloth, or similar material.

As shown in FIG. 12B, the electrodes 23 are located on the outer periphery of the electrode patch 40 with a bottom surface of the electrodes configured to come into contact with the patient's skin. In this embodiment, the ECG data acquisition module 41 and the electrode patch 40 are disposable, and the data acquisition module 41 of wireless ECG module 15 includes embedded circuitry 112. The embedded circuitry includes, for example, the data acquisition circuit 26, microcontroller 28, on-board memory 30, the rechargeable battery 27, and patient alarm 29, as described with reference to FIG. 3.

As shown in FIG. 12B, electrical connection is establish between electrodes 23 of the electrode patch 40 and the embedded circuitry 112 by wires or a flexible circuit 110 embedded in the wireless ECG module 15. The electrical connection allows for ECG signals from the ECG electrodes 23 to be transmitted to the embedded circuit 110 of the ECG data acquisition module 41.

The embodiment of FIGS. 12A and 12B of the wireless ECG module 15 can simplify the interconnection design and would eliminate the requirement for a rechargeable power source (i.e. having to swap out between two ECG data acquisition modules). From a clinical perspective, a totally disposable device can improve the patient's outcome by reducing the risk of infection which at times may be caused from improper cleaning methods associated with reusable devices.

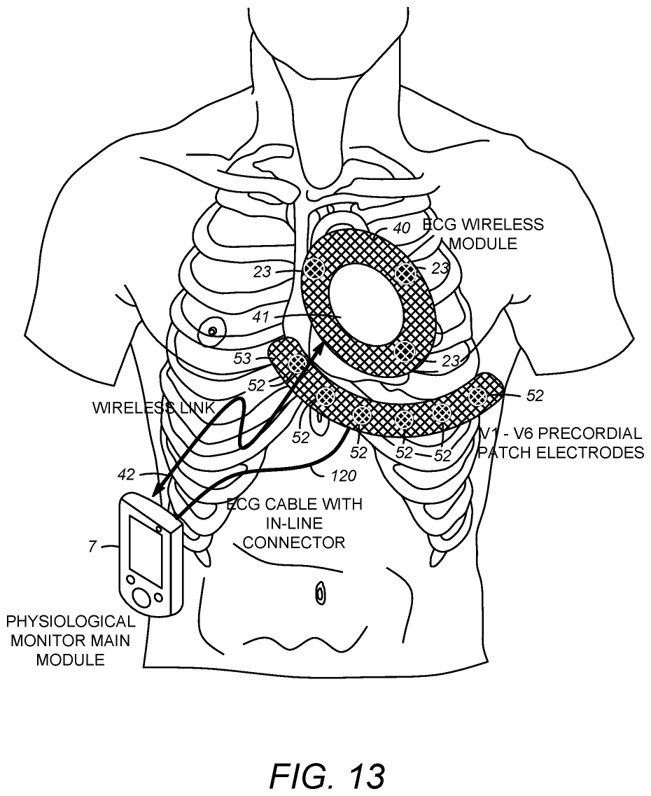

FIG. 13 is a diagram of a physiological monitoring system including the precordial electrode array connected to the main module according to an embodiment of the present disclosure.

The physiological monitoring system of FIG. 13 differs from the physiological monitoring system of FIG. 6 in that the precordial electrode array (or set) 53 is connected to the main module 7 instead of being connected to the wireless ECG module 15. The precordial electrode array 53 (e.g., 5 or more electrodes) can be placed over the precordial locations of the right and left chest proximate to the ECG wireless module 15. The precordial electrode array 53 is composed of a breathable porous material integrated with the electrodes 52. However, the precordial electrode array 53 can also be composed of silicon, polymer, foam, cloth, or similar material.

The precordial electrode array 53 is attached to the patient using, for example, a biocompatible adhesive or an adhesive surface on the bottom surface of the precordial electrode array 53 facing the patient's skin. However, the precordial electrode array 53 can be made from a material that is self-adhesive.

The precordial electrode array 53 is connected to the main module 7 by a cable 120 inserted into an external connection port (e.g., cable port) in the main module 7. The precordial electrode set 53 includes a cable 120 that terminates with an in-line connector. The in-line connector is received in the external connection port of the main module 7, thereby establishing a connection between the precordial electrode array 53 and the circuitry of the main module 7. This configuration provides the capability of obtaining additional ECG data for a diagnostic data similar to the 12 lead recording without having to remove the primary electrodes (e.g., 3) of the wireless ECG module 15. Once the additional 12 lead ECG data is obtained, the precordial electrode array 53 can be disconnected after it is determined that the additional diagnostic ECG data is no longer required.

The cable 120 between the precordial electrode set 53 and the main module 7 is, for example, an electrical cable or other similar interface cable. Once attached, the electrical signals from the electrodes 52 of the precordial electrode array 53 are received by the main module 7, the data signals are processed by the circuitry of the main module 7 as described previously with reference to FIGS. 1 and 2.

For example, the data signals from the electrodes 52 are input to an ECG data acquisition circuit 9, which includes amplifying circuitry, filtering circuitry, and A/D circuitry that convert the analog signals to digital signals using amplification, filtering, and A/D conversion methods known in the art. The processing of the ECG data signals by the ECG data acquisition circuit 9 produces digital data waveforms, which are passed to a microcontroller 12, which analyzes the digital waveforms to identify certain digital waveform characteristics and threshold levels indicative of abnormal conditions of the patient. The microcontroller 12 can identify any abnormal cardiac conditions (e.g. arrhythmias, or ST segment measurements indicative of ischemia or myocardial infarction).

Additionally, the microcontroller 13 includes communication interface circuitry for establishing communication connections with various devices and networks using both wired and wireless connections for transmitting physiological data, results of the analysis by the microcontroller 12, and alerts and/or alarms to the patient, clinicians and caregivers regarding any abnormal conditions detected as well as storing the physiological data in the on-board memory 22.

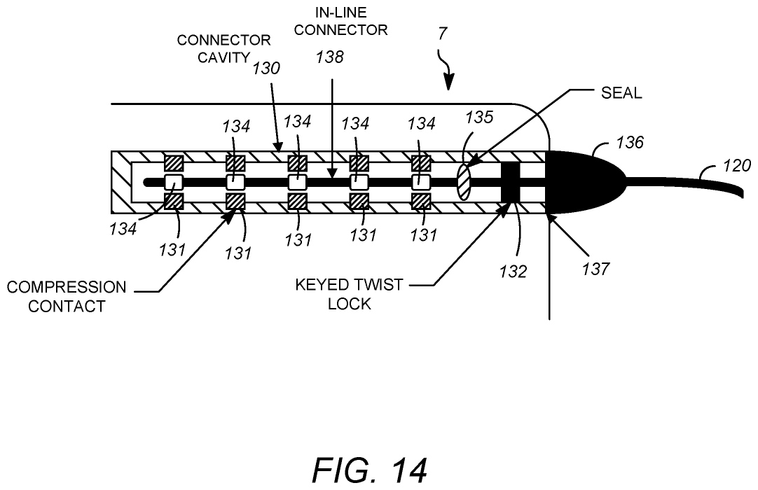

FIG. 14 is a side view of the connection between the main module and the precordial electrode array according to an embodiment of the present disclosure.

The precordial electrode array 53 includes a cable 120 that terminates with an in-line connector 138. As shown in FIG. 14, the in-line connector 138 includes a series of electrical contacts 134 that are received in the cable port 137 of the main module 7. The cable port 137 includes a connector cavity 130 having a series of compression contacts 131 that align with the series of electrical contacts 134 when the in-line connector 138 is fully inserted into the connector cavity 130 of the main module 7. When the in-line connector 138 is fully inserted into the connector cavity 130 of the main module 7, the in-line connector 138 establishes an electrical connection with the circuitry of the main module 7.

The in-line connector 138 also includes an integrated seal 135 that is, for example, concentric around the ECG cable 120 for providing a water tight seal, thereby protecting the integrity of the electrical connection between the and the precordial electrode array 53 and the main module 7 when the in-line connector 138 is received in the cable port 137. Having the seal 135 integrated within the ECG cable 120 rather than having an O-ring within the connector cavity 130 can improve the reliability of the seal 135 by avoiding a multi-use configuration.

The in-line connector 138, once establishing a connection between the main module 7 and the precordial electrode array 53, is held securely in place using a lock mechanism such as a keyed twist lock 132. The keyed twist lock 132 is inserted in the ECG cable port 137 of the main module 7 and then twisted (e.g., 90°) and aligned within a grooved slot (e.g., in the connector cavity). The In-line connector 138 is guided into cable port 137 and the connector cavity 130 (i.e., for establishing a connection between the main module 7 and the precordial electrode array 53 by holding a strain relief portion 136 of the in-line connector 138, which prevents damage to the precordial electrode array 53 and the cable 120. The strain relief portion 136 is integrated into the ECG cable 120, which can also provide additional protection from water ingress.

The subject matter described in the present disclosure of the present application provides many technical improvements over conventional patient monitoring devices and systems that includes, for example, improved outcomes of patients during ambulatory activity associated with their recuperation and rehabilitation by providing a simplified ECG electrode placement, a detachable precordial electrode array, wireless communications between product subsystems which eliminates wires between ECG electrodes and physiological monitor, and improved performance with respect to noise immunity and reliability.

Technical improvements over conventional patient monitoring devices and systems also include interconnections of the electrode array to the main module and the wireless ECG electrode array, which can be configured with an in-line connection. Additionally, the electrode array and the wireless ECG module have the capability to adjust individual electrode locations which could allow for application specific cardiac vectors to each patient. Moreover, the wireless ECG module can include disposable components and circuitry.

The present disclosure may be implemented as any combination of an apparatus, a system, an integrated circuit, and a computer program on a non-transitory computer readable recording medium. The microcontrollers may be implemented as an integrated circuit (IC), an application specific integrated circuit (ASIC), or large scale integrated circuit (LSI), system LSI, super LSI, or ultra LSI components which perform a part or all of the functions of the wireless ECG module and main module.

Each of the components of the wireless ECG module and the main module of the present disclosure can be implemented using many single-function components, or can be one component integrated using the technologies described above. The various illustrative circuits and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The present disclosure includes the use of computer programs or algorithms in the wireless ECG module and the main module. The programs or algorithms can be stored on a non-transitory computer-readable medium for causing a computer, such as the microcontroller, to execute the steps described in FIGS. 2 and 4. The computer programs, which can also be referred to as programs, software, software applications, applications, components, or code, include machine instructions for a programmable processor, and can be implemented in a high-level procedural language, an object-oriented programming language, a functional programming language, a logical programming language, or an assembly language or machine language. The term computer-readable recording medium refers to any computer program product, apparatus or device, such as a magnetic disk, optical disk, solid-state storage device, memory, and programmable logic devices (PLDs), used to provide machine instructions or data to a programmable data processor, including a computer-readable recording medium that receives machine instructions as a computer-readable signal.

By way of example, computer-readable medium can comprise RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, include compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The subject matter of the disclosure of the present application are merely provided as examples of patient monitoring devices and systems. Further features or variations are contemplated in addition to the features of the patient monitoring apparatus and systems described above. It is contemplated that the implementation of the components of the present disclosure can be done with any newly arising technology that may replace any of the above implemented technologies.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments.

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” indicates an example or instance and does not imply or require any preference for the noted example. Thus, the disclosure is not to be limited to the examples and designs described herein but is to be accorded the widest scope consistent with the principles and novel features disclosed herein.

What is claimed is:

1. A physiological monitoring system for providing monitoring of a patient, comprising:
an electrocardiogram (ECG) module including an ECG microcontroller and a first plurality of electrodes worn on the patient, the ECG microcontroller being coupled to the first plurality of electrodes for receiving first physiological data gathered by the first plurality of electrodes;
a main module detachably worn by the patient and connected to the ECG module by a first communication connection, the main module including a main controller, an on-board memory, and a communication interface coupled to the main controller,
the main controller being configured to:
receive the first physiological data from the ECG module using the first communication connection.

2. The physiological monitoring system of claim 1, wherein the ECG microcontroller is further configured to:
analyze the first physiological data gathered by the first plurality of electrodes,
identify one or more abnormal conditions of the patient, and
transmit in real-time results of the analysis of the first physiological data and the identified abnormal conditions to the main module using the first communication connection.

3. The physiological monitoring system of claim 1 or 2, further comprising a detachable precordial electrode array including a second plurality of electrodes worn in a precordial location of the patient proximate to the ECG module, the second plurality of electrodes being configured to gather second physiological data,
the detachable precordial electrode array being connected to the ECG module by a second communication connection.

4. The physiological monitoring system of claim 3, wherein the ECG microcontroller is further configured to:
analyze the second physiological data gathered by the second plurality of electrodes,
identify one or more abnormal conditions of the patient, and
transmit in real-time results of the analysis of the second physiological data and the identified abnormal conditions to the main module using the first communication connection.

5. The physiological monitoring system of claim 4, wherein the ECG microcontroller is further configured to transmit an alert to the patient based on the analysis of the first physiological data or the second physiological data.

6. The physiological monitoring system of claim 4, wherein the main controller is further configured to:
transmit in real-time results of the analysis of the first physiological data or the second physiological data and the identified abnormal conditions using a first wireless protocol of the communication interface, or
store the first physiological data or the second physiological data and the identified abnormal conditions in the on-board memory when the main module is unable to transmit the in real-time using the first wireless protocol of the communication interface.

7. The physiological monitoring system of claim 6, wherein the main controller is further configured to:
transmit in real-time results of the analysis of the first physiological data or the second physiological data and the identified abnormal conditions using a second wireless protocol of the communication interface if a significant physiological event is identified from the analysis of the first physiological data or the second physiological data.

8. The physiological monitoring system of claim 6, wherein the first wireless protocol is in accordance with WIFI or Bluetooth.

9. The physiological monitoring system of claim 7, wherein the second wireless protocol is in accordance with a cellular network.

10. The physiological monitoring system of claims 1, 2 or 4, wherein the first communication connection is a wireless connection.

11. The physiological monitoring system of claim 3, wherein the second communication connection is a wired connection.

12. The physiological monitoring system of claim 1, wherein the ECG module further comprises a data acquisition module with embedded circuitry.

13. The physiological monitoring system of claim 12, wherein second communication connection includes an in-line connector detachably connected to the data acquisition module

14. The physiological monitoring system of claim 12, wherein the data acquisition module is composed of a flexible material.

15. The physiological monitoring system of claims 12 or 14, wherein the first plurality of electrodes are formed in a flexible material integrated as a patch having a top surface that is attachable to the data acquisition module and a bottom surface that is attachable to the patient.

16. The physiological monitoring system of claim 15,
wherein the patch with the first plurality of electrodes is detachable from the data acquisition module and disposable, and
the data acquisition module is re-useable.

17. The physiological monitoring system of claim 15, wherein the data acquisition module and the patch with the first plurality of electrodes are integrated, and both the patch and the data acquisition module are disposable.

18. The physiological monitoring system of claim 1, wherein the ECG module further comprises adjustment slots and a location of each of the first plurality of electrodes is adjustable within the adjustment slots.

19. The physiological monitoring system of claim 3, wherein the detachable precordial array further comprises adjustment slots and a location of each of the second plurality of electrodes is adjustable within the adjustment slots.

20. The physiological monitoring system of claim 3, wherein the detachable precordial array is composed of a flexible material.

21. The physiological monitoring system of claim 1, wherein the first physiological data gathered by the first plurality of electrodes includes ECG data.

22. The physiological monitoring system of claim 3, wherein the second physiological data gathered by the second plurality of electrodes includes ECG data.

23. The physiological monitoring system of claim 22, wherein the first plurality of electrodes and the second plurality of electrodes include wired sensors for gathering respective first and second physiological data.

24. The physiological monitoring system of claim 1, wherein the main module further comprises a plurality of electrodes for gathering physiological data that includes ECG data.

25. The physiological monitoring system of claim 1, wherein the monitoring provided by the physiological monitoring system is continuous.

26. The physiological monitoring system of claim 1, further comprising a detachable precordial electrode array including a second plurality of electrodes worn in a precordial location of the patient proximate to the ECG module, the second plurality of electrodes being configured to gather second physiological data,
the detachable precordial electrode array being connected to the main module by a third communication connection for transmitting the second physiological data gathered by a second plurality of electrodes to the main module,
wherein the main controller is further configured to:
receive the second physiological data from the ECG module using the third communication connection.

27. The physiological monitoring system of claim 26, wherein the main controller is further configured to:
analyze the second physiological data gathered by the second plurality of electrodes, and
identify one or more abnormal conditions of the patient.

28. The physiological monitoring system of claim 27, wherein the main controller is further configured to:
transmit in real-time results of the analysis of the second physiological data and the identified abnormal conditions using a first wireless protocol of the communication interface, or
store the second physiological data and the identified abnormal conditions in the on-board memory when the main module is unable to transmit the in real-time using the first wireless protocol of the communication interface.

29. The physiological monitoring system of claim 28, wherein the main controller is further configured to:
transmit in real-time results of the analysis of the first physiological data or the second physiological data and the identified abnormal conditions using a second wireless protocol of the communication interface if a significant physiological event is identified from the analysis of the first physiological data or the second physiological data.

30. The physiological monitoring system of claim 28, wherein the first wireless protocol is in accordance with WIFI or Bluetooth.

31. The physiological monitoring system of claim 29, wherein the second wireless protocol is in accordance with a cellular network.

32. The physiological monitoring system of claim 26, wherein the first communication connection is a wireless connection.

33. The physiological monitoring system of claim 26, wherein the third communication connection is a wired connection.

34. The physiological monitoring system of claim 33, wherein the third communication connection includes an in-line connector detachably connected to the main module.

35. An electrocardiogram (ECG) module for providing monitoring of a patient, the ECG module comprising:
a plurality of electrodes worn on the patient and configured to gather first physiological data;
a data acquisition module including embedded circuitry;
a first communication connection; and
an ECG microcontroller coupled to the plurality of electrodes, the data acquisition module, and the first communication connection,
the ECG microcontroller being configured to:
receive the first physiological data gathered by the plurality of electrodes; and
transmit in real-time the first physiological data using the first communication connection.

36. The ECG module of claim 35, wherein the ECG microcontroller is further configured to:
analyze the first physiological data gathered by the plurality of electrodes,
identify one or more abnormal conditions of the patient, and
transmit in real-time results of the analysis of the first physiological data and the identified abnormal conditions using the first communication connection.

37. The ECG module of claim 36, wherein the ECG microcontroller is further configured to transmit an alert to the patient based on the analysis of the first physiological data.

38. The ECG module of claim 35, wherein the data acquisition module is composed of a flexible material.

39. The ECG module of claim 35, wherein the plurality of electrodes are formed in a flexible material integrated as a patch having a top surface that is attachable to the data acquisition module and a bottom surface that is attachable to the patient.

40. The ECG module of claim 39,
wherein the patch with the plurality of electrodes is detachable from the data acquisition module and disposable, and
the data acquisition module is re-useable.

41. The ECG module of claim 39, wherein the data acquisition module and the patch with the plurality of electrodes are integrated, and both the patch and the data acquisition module are disposable.

42. The ECG module of claims 35 or 36, wherein the first communication connection is a wireless connection.

43. The ECG module of claim 35, further comprising a second communication connection connectable to a detachable precordial electrode array for receiving second physiological data.

44. The ECG module of claim 43, wherein the ECG microcontroller is further configured to:
analyze the second physiological data,
identify one or more abnormal conditions of the patient, and
transmit in real-time results of the analysis of the second physiological data and the identified abnormal conditions using the first communication connection.

45. The ECG module of claim 44, wherein the ECG microcontroller is further configured to transmit an alert to the patient based on the analysis of the second physiological data.

46. The ECG module of claim 43, wherein the second communication connection is a wired connection.

47. The ECG module of claim 46, wherein the second communication connection includes an in-line connector detachably connected to the data acquisition module.

48. The ECG module of claim 35, further comprising adjustment slots, wherein a location of each of the plurality of electrodes is adjustable within the adjustment slots.

49. The ECG module of claim 35, wherein the first physiological data gathered by the plurality of electrodes includes ECG data.

50. The ECG module of claim 35, wherein the plurality of electrodes include wired sensors for gathering the first physiological data.

51. The ECG module of claim 35, wherein the monitoring provided by the ECG module is continuous.

52. A detachable precordial electrode array, comprising:
a plurality of electrodes worn in a precordial location of a patient, the plurality of electrodes being configured to gather physiological data; and
a communication connection for transmitting the physiological data,
wherein the plurality of electrodes are formed in a flexible material integrated as a patch and having a bottom surface that is attachable to the patient for gathering the physiological data.

53. The detachable precordial electrode array of claim 52, wherein the communication connection is a wired connection.

54. The detachable precordial electrode array of claims 52 or 53, wherein the communication connection includes an in-line connector.

55. The detachable precordial electrode array of claim 52, wherein the detachable precordial electrode array further comprises adjustment slots and a location of each of the plurality of electrodes is adjustable within the adjustment slots.

56. A physiological monitoring system for providing monitoring of a patient, comprising:
an electrocardiogram (ECG) monitoring module including an ECG control means and a ECG physiological data gathering means worn in a precordial location of the patient, the ECG control means being coupled to the ECG physiological data gathering means for receiving physiological data;
a main monitoring module detachably worn by the patient and connected to the ECG monitoring module by a communication connection, the main monitoring module including a main control means and a communication means coupled to the main control means,
the main control means being configured to:
receive the physiological data from the ECG monitoring module using the communication connection, and
transmit in real-time the physiological data using the communication means.

57. The physiological monitoring system of claim 56, wherein the communication connection is a wireless connection.

58. The physiological monitoring system of claim 56, wherein the ECG control means is further configured to transmit an alert to the patient based on the physiological data.
